(19) United States
(12) Patent Application Publication
(10) Pub. No.: US 2014/0178507 A1
(43) Pub. Date: Jun. 26, 2014

(54) WEIGHT MANAGEMENT COMPOSITION

(71) Applicant: CRAVING CRUSHER, LLC, Miami, FL (US)

(72) Inventor: Om AGARWALA, Seattle, WA (US)

(73) Assignee: CRAVING CRUSHER, LLC, Miami, FL (US)

(21) Appl. No.: 13/723,987

(22) Filed: Dec. 21, 2012

(51) Int. Cl. A61K 38/01; A61K 36/24; A61K 36/33; A61K 31/196; A61K 36/38; A61K 31/685; A61K 36/53; A61K 36/888; A61K 36/82

(52) U.S. Cl. CPC A61K 38/018; A61K 36/888; A61K 36/24; A61K 36/33; A61K 36/82; A61K 36/38; A61K 31/685; A61K 36/53; A61K 31/196
USPC 424/727; 514/783; 514/785; 514/777

(57) ABSTRACT

A composition for oral administration for weight management includes at least approximately ⅔ (by weight) active or nutritional ingredients, and may include as much as and approximately ⅓ (by weight) of sweetening, flavoring and/or palatability ingredients. The composition is free of caffeine and free of added sugars.

### FIELD OF THE DISCLOSURE

The present disclosure relates to a weight management composition. The present disclosure particularly relates to a composition for weight management through the approach of appetite suppression based on a subject' sense of satiety.

### BACKGROUND:

Obesity is a chronic, complex, multi-factorial disease, involving social, cultural, genetic, physiological and psychological components and is associated with substantially increased morbidity and mortality.

The incidence of obesity in the general population of the United States has shown a dramatic increase over the last decade with over 50% of the population considered overweight or obese. A similar trend is observed in other countries as the so-called Western diet is adopted.

Environmental and behavioral changes brought about by economic development and modernization has been linked to the rise in global obesity. The environmental factors which foster the tendency toward obesity include lack of physical activity combined with high-calorie foods. The prevalence of overweight and obesity is increasing worldwide at an alarming rate in both developing and developed countries, in children and adults, men and women.

Obesity is significantly associated with increase in the risk of various diseases such as high blood pressure, diabetes, osteoarthritis, heart disease, stroke, gallbladder disease and cancers of the breast, prostate and colon. Weight gain has also been found to occur as a result of various factors, including, for example, use of certain drugs, cessation of smoking, and advent of a holiday season.

Drugs which are known to cause weight gain include antipsychotics, particularly atypical antipsychotics; antidepressants, particularly the tricyclic antidepressants; mood-stabilizers; calcium channel blockers; anti-convulsants; proton pump inhibitors; antidiabetic agents; antihypertensives; and hormones. Weight gain associated with use of certain drugs may significantly affect patient compliance with the drug administration regime. Certain drug categories, such as the SSRIs and tricyclic antidepressants, cause food cravings.

There are several different treatment options for management of weight, including: dietary therapy, physical activity, behavior therapy, drug therapy and surgery. For the majority of overweight and obese people, who find they are unable to change their lifestyle, drug therapy is the most favorable and applicable option. Although hundreds of millions of people are seeking drug therapy for the treatment of obesity, current drug therapies do not meet this need due to their undesired side effects and limited efficacy. Medications currently approved for weight loss in the United States fall into two categories: those that decrease food intake by reducing appetite or increasing satiety (appetite suppressants), and those that decrease nutrient absorption.

A number of approaches have been proposed or used to help subjects reduce food intake, otherwise referred to as energy intake, and thereby manage their body weight. These approaches include use of agents to act on the central nervous system to increase levels of serotonin, and those acting in the gastrointestinal tract to reduce digestion and/or absorption of nutrients. These approaches suffer from potential side effects that reduce their utility for long-term body weight management.

Current nutritional recommendations often include the recommendation to consume a high fiber diet or to consume foodstuffs that are high in dietary fiber. However, high fiber diets are often associated with several undesirable side effects including decreased palatability of food, increased stool volume, increased defecation frequency, poor skin and hair, improper mineral balance and decreased food digestibility. Unfortunately, even though these types of nutritional recommendations have existed for many years, the prevalence of obesity in humans has continued to increase. The relative abundant access to foods and the dietary habits of the population continues to make it a challenge for humans to lose weight.

Another nutritional recommendation that is often made concerning weight management is to reduce dietary intake of fats. Although numerous low fat, no fat, reduced fat and “light” types of foodstuffs have appeared and increased their presence in the human food supply, the prevalence of obesity has continued to increase.

Accordingly, there is still a need in the present art to develop a composition that assists humans in reducing body fat and overall weight.

### SUMMARY

In accordance with one aspect of the present disclosure there is provided a composition for weight management; said composition comprising active ingredients and “flavoring” ingredients, the “flavoring” ingredients providing palatability, the composition including one or more of the following:
a) a source of protein;
b) coconut water;
c) nopal pad extract;
d) Caralluma fimbriatta;
e) tea extract;
Garcinia cambogia extract;
g) N-oleayl-phosphatidyl-ethanolamine (OEA)
h) epigallocatechin gallate (EGCG);
i) vitamin B-3;
coleus forshkohlii (extract);
k) optionally a sweeting ingredient;
l) optionally flavoring and/or palatability ingredients such as at least one ingredient selected from citric acid, coconut milk powder, ginger powder, xanthan gum, cherry juice concentrate; strawberry juice concentrate, chocolate powder, cocoa powder; and
m) optionally at least one pharmaceutically acceptable excipient selected from the group consisting of disintegrants, glidents, diluents, binders, emulsifying agents, and suspending agents.

It should be appreciated that the optional ingredients utilized for flavoring and palatability may include one or more of:
a) Fruit and/or vegetable juices such as tomato juice, apple juice, pear juice, cherry juice, dragon juice, elderberry juice, passion fruit juice, pomegranate juice, strawberry juice, etc.;
b) Organic acids such as acetic, citric, malic, tartaric and phosphoric;
c) Emulsifying and suspending agents such as guar, xanthan, pectin and alginates; and
d) Other flavoring agents such as cocoa, coffee and caramel.

It should further be appreciated that a preferred form of the weight management composition does not include the addition of sugar.

Ranges and/or amounts of ingredients will be presented in the various examples below. The following table is provided as a non-limiting example.

TABLE 1
The following table presents non-limiting examples of the ranges of
the ingredients if present, with the understanding that not all listed
ingredients need be present in the weight management composition.

| | Lower end of concen- | Upper end of concen- |
|---|---|---|
| | tration range as % | tration range as % |
| | of total mass of the | of total mass of the |
| | composition if the | composition if the |
| Ingredient | ingredient is present | ingredient is present |
| Source of protein such as | About 10% | About 20% |
| whey protein hydrolyzate | | |
| Coconut water | About 1% | About 35% |
| Nopal pad extract if in | About 0.1% | About 1.0% |
| the form of nopal freeze | | |
| dried powder | | |
| Nopal pad extract if in | About 15% | About 30% |
| the form of nopal juice | | |
| Caralluma fimbriatta | About 0.1% | About 1.0% |
| Tea extract such as | About 0.01% | About 0.5% |
| greent tea extract | | |
| Garcinia cambogia | About 0.3% | About 1.0% |
| extract | | |
| OEA + EGCG | About 0.5% | About 2.0% |
| Vitamin B-3 | About 0.01% | About 0.5% |
| Citric acid | About 0.5% | About 2.0% |
| Coconut milk powder | About 0.5% | About 2.0% |
| Ginger powder | About 0.01% | About 0.5% |
| Cherry juice concentrate | About 15% | About 25% |
| Strawberry juice | About 10% | About 15% |
| concentrate | | |
| Chocolate powder or | About 0.5% | About 1.5% |
| cocoa powder | | |
| Rebaudinoside | About 0.05% | About 2.0% |
| Xanthan gum | About 0.02% | About 1.0% |
| Coleus forshkohlii | About 0.05% | About 0.50% |
| (10% extract) | | |

Preferably the composition is formulated for oral administration as a liquid or beverage with a pH in the range of 2.0 to 9.0, however, the pH range as just described should be considered a non-limiting example.

The composition may alternatively be formulated for oral administration into a dosage form such as a powder, a tablet, a pill, a capsule, a liquid, a beverage, a “shake”, a suspension, an emulsion and/or in the form of a powder for re-constitution.

In accordance with another aspect of the present disclosure there is provided a method for weight management, said method comprising administering a therapeutically effective amount of composition to a subject.

The composition may be administered to a subject in single and plurality of doses or servings daily over the course of a weight management program. Since the purpose of the composition is to provide for weight management, it is preferred that the composition be taken about 0.5 to 2 hours before meals, although it should be appreciated that as an alternative the composition may be taken with consumption of a lesser amount of food than would ordinarily be taken by the subject. Thus, for example, a subject may take the composition in lieu of food and the composition may satiate any hunger craving for two or more hours.

Typically, the total deliverable dosage of the composition to a subject is from about 1.0 g per kg body weight to about 10.0 g per kg body weight, it being understood, however, that the foregoing range is illustrative and non-limiting.

### DESCRIPTION

Various formulations containing herbal ingredients or phyto-constituents have been disclosed in the prior art documents. These known formulations produce moderate to average weight control effect but do not provide for management of weight for a prolong period of time.

In non-scientific, subjective studies of taste and efficacy, persons before taking a composition of the type described, when asked to rate their level of hunger on a subjective scale of 1 to 10 with 10 being the highest level of hunger) had attained a hunger average of more than 8. Those same persons when asked to rate their satiety (or lack of hunger) on a subjective scale of 1 to 10 (with 10 being the highest degree of satiety or lowest degree of hunger) two hours after taking the composition of the type described in an amount of 74 ml (approximately 2.5 ounces) had attained a satiety average of more than 8.

The weight management composition as disclosed includes ingredients for efficacy and ingredients for flavoring and palatability.

Contrary to typical weight management or appetite suppressant products commercially available, the present weight management composition is free of caffeine and preferable has no sugar added although it is to be recognized that naturally occurring sugar is present in various flavoring ingredients such as fruit juices.

The weight management composition of the present disclosure produces the desired effects (weight management) by a combination of central and peripheral mechanisms such as:
regulating appetite and reducing the amount of fat;
inducing a stronger thermogenic effect, the results of which may not be clearly understood, but which may (a) increase the rate at which calories are used by the body and/or (b) temperature sensitive neurons in the hypothalamus get stimulus to control the brain's food intake circuitry;
reducing appetite by acting on hunger hormones; and
increasing the conversion of food (carbohydrate) into glycogen and suppressing de novo fatty acid synthesis.

The components or constituents of the weight management composition as described herein may function as follows, it being understood, however, that not all components or constituents are necessarily present in the composition.

A source of protein: Proteins such as whey protein hydrolyzate has a fast absorption rate in the gastrointestinal tract which induces a stronger thermogenic. Furthermore, glycomacropeptide of whey protein hydrolyzate is a powerful stimulator of cholecystokinin receptor (CCK) which in turn suppresses appetite. Other sources of protein may be used either in combination with whey protein, or instead of whey protein, or in combination with each other—examples include whey, whey protein concentrate, whey protein isolate, whey protein hydrolyzate, milk protein concentrate, calcium or sodium caseinate, soy protein isolate, gelatin, collagen, potato protein, soy and egg proteins. In this regard it is beneficial to take into account dietary intolerances of consumers, e.g., lactose intolerance and/or the need for a gluten free protein.

Coconut water is used in the composition of the present disclosure to balance the potassium, magnesium and other electrolytes in the body.

Laurie acid, a C12 fatty acid, present in the coconut milk reduces appetite by acting on hunger hormones.

Nopal cactus has been a staple part of Mexican and Tex-Mex cuisine for centuries. The nopal extract contains vitamins A, B-1, B-2, B-3, and C. Additionally, nopal extract is a good source of both insoluble and soluble fiber. The high fiber content of nopal helps to regulate appetite and reduce the amount of fat in the body by helping the breakdown of fat. In the present composition, the nopal extract may be used in a form of a nopal freeze dried powder or alternatively the nopal extract is used in a form of a nopal juice.

Caralluma fimbriatta present in the composition prevents fat accumulation via blocking citrate lyase. It is an edible succulent cactus that belongs to the family Asclepiadaceae. Caralluma plants have been reported to possess medicinal properties like anti-diabetic, anti-inflammatory, antiobesity etc. which have been attributed to their chemical constituents, particularly glycosides belonging to pregnane group. Its key ingredients ate pregnane glycosides, flavone glycosides, megastigmane glycosides, bitter principles, saponins and various other flavonoids.

The appetite suppressant action of C. fimbriatta could be mainly attributed to the pregnane glycosides. These compounds seem to have peripheral and central effects. In the adipose tissue, pregnane glycosides reduce lipogenesis. The extract of C. fimbriatta is a competitive inhibitor of ATP-citrate lyase, an extra mitochondrial enzyme involved in the initial 25 steps of de novo lipogenesis. C. fimbriatta also contains hydroxycitrate (HCA) as active component. HCA has also been reported to cause weight loss in humans without stimulating the central nervous system consequently, HCA further reduces the transformation of citrate into acetyl coenzyme A, a step necessary for the formation of fatty acids in the liver.

It is known that the long term consumption of teas and their extracts, such as green tea and its extract (GTE) have been associated with weight loss mainly through a thermogenic mechanism. The main active ingredients in GTE such as catechins epigallocatechin gallate, epigallocatechin (EGC), epicatechin gallate (ECG), and epicatechin (EC) are responsible for many of the beneficial effects of green tea.

Garcinia Cambogia may promote weight reduction through suppressed de novo fatty acid synthesis, increased lipid oxidation and reduced food intake. It increases the conversion of food (carbohydrate) into glycogen. It works to slow down the production of fat and burns the fat which is already accumulated. From the available literature, Garcinia Cambogia may be more effective at moderating weight gain than at promoting weight loss, making the compound potentially more useful for weight maintenance after an initial loss.

Ginger present in the composition enhances the thermic effect of food and promotes feeling of satiety without affecting metabolic and hormonal parameters.

N-oleayl-phosphatidyl-ethanolamine (OEA) is used to increase fat oxidation.

Epigallocatechin gallate (EGCG), may be added or included as in ingredient in green tea extract as described above.

Vitamin B-3 (niacin) provides the function of suppressing appetite, delaying hunger and also to regulating blood sugar levels. Xanthan gum functions to curb the appetite.

It should be appreciated that the precise mechanism of the present composition may not be known with certainty but the descriptions above are based on the best available knowledge.

It should further be appreciated that a sweetening ingredient may be used such as Rebaudioside (also referred to as Rebaudioside A or “Reb A” or Rebiana A—a natural sweetener which does not meaningfully affect the caloric value of the present weight management composition. Reb A is 200 times sweeter than sugar. Reb A provides 0.2 calories pet gram while natural sugar provides 2.9 calories per gram. If, for example 0.1 grams of natural sugar was to be used in the weight management composition, the natural sugar would contribute 0.29 calories. Since Reb A is 200 times sweeter than natural sugar, the same sweetening effect would be achieved by using 0.1/200 grain (0.0005 grams) of Reb A which would contribute only 0.0001 calories to one gram of the weight management composition. Thus Reb A also known as stevia, as well as aspertame, sucralose and sugar alcohols, all of which are recognized as non-nutritive and/or essentially calorie free may be used.

The weight management composition preferably includes flavoring and/or palatability agents. Fruit juices, vegetable juices, organic acids, emulsifying and suspending agents may be used so that the weight management composition may be modified to accommodate taste preferences of the users.

The composition may be administered to the subject in single or plurality of doses or servings daily over the course of a weight management program but should not be considered a complete substitute for food.

Approximately ⅔ of the weight management composition described herein (weight % by mass) includes active ingredients and approximately ⅓ of the weight management composition includes sweetening, flavoring and palatability ingredients. It should be further recognized, however, that to accommodate the taste preferences of individuals, these proportions may vary somewhat.

There are, on the market, various compositions which purport to provide nutritional benefits, energy benefits, etc., and many of these are marketed in liquid form within containers (bottles) of 74 ml (2.5 ounces). Certain information is frequently provided such as ingredients and the calories in the composition. Therefore, the following information is provided with the understanding that this is exemplary and non-limiting. Consuming 74 milliliters (2.5 ounces) of the weight management composition described herein is estimated to provide approximately the following:

| Calories | 40 | |
|---|---|---|
| Calories from Fat | 0 | |
| Total Carbohydrates | 3 | g |
| Dietary Fiber | 1 | g |
| Sugars | 2 | g |
| Protein | 8 | g |
| Vitamin A | 50 | IU |
| Vitamin B-3 (as niacin) | 4 | mg |
| Vitamin C | 3 | mg |
| Sodium | 5 | mg |
| Potassium | 30 | mg |

The following are non-limiting examples of the weight management composition.

### EXAMPLE 1

A liquid composition is prepared using the following ingredients and approximate amounts.

| | % with respect to | Milliliters per |
|---|---|---|
| | the total mass of | 74 milliliter |
| INGREDIENTS | the composition | composition |
| whey protein hydrolyzate | 12.00 | 8.88 |
| coconut water concentrate | 30.00 | 22.20 |
| nopal extract | 21.95 | 16.24 |
| Caralluma fimbriatta | 0.200 | 0.15 |
| green tea extract | 0.05 | 0.035 |
| Garcinia cambogia extract | 0.60 | 0.44 |
| a mixture of N-oleayl-phosphatidyl- | 0.15 | 0.105 |
| ethanolamine (OEA) and | | |
| epigallocatechin gallate (EGCG) | | |
| vitamin B3 | 0.05 | 0.04 |
| citric acid | 0.80 | 0.600 |
| coconut milk powder | 1.00 | 0.74 |
| ginger powder | 0.05 | 0.04 |
| xanthan gum | 0.05 | 0.04 |
| cherry juice concentrate | 19.10 | 14.13 |
| strawberry juice concentrate | 12.80 | 9.47 |
| chocolate powder | 1.00 | 0.74 |
| Rebaudioside | 0.15 | 0.11 |
| Coleus forskholii 10% extract | 0.05 | 0.04 |
| Total | 100 | 74.00 |

### EXAMPLE 2

A liquid composition is prepared using the following ingredients and approximate amounts.

| | % with respect to the total |
|---|---|
| INGREDIENTS | mass of the composition |
| Water | 77.50 |
| whey protein hydrolyzate | 12.00 |
| coconut water concentrate | 2.00 |
| Nopal freeze dried powder | 0.30 |
| Caralluma fimbriatta | 0.200 |
| green tea extract | 0.05 |
| Garcinia cambogia extract | 0.60 |
| a mixture of N-oleayl-phosphatidyl- | 0.20 |
| ethanolamine (OEA) and | |
| epigallocatechin gallate (EGCG) | |
| vitamin B3 | 0.05 |
| citric acid | 0.80 |
| coconut milk powder | 1.00 |
| ginger powder | 0.05 |
| Coleus forskholii 10% extract | 0.05 |
| xanthan gum | 0.05 |
| cherry juice concentrate | 3.00 |
| strawberry juice concentrate | 1.00 |
| chocolate powder | 1.00 |
| rebaudioside | 0.15 |
| Total | 100 |

### EXAMPLE 3

| | % with respect to | Milliliters per |
|---|---|---|
| | the total mass of | 74 milliliter |
| INGREDIENTS | the composition | composition |
| Pomegranate juice from concentrate | 33.300 | 24.64 |
| Tender Coconut water from | 30.000 | 22.20 |
| concentrate | | |
| Nopal pad juice from powder | 21.95 | 16.24 |
| Whey protein hydrolyzate | 12.000 | 8.88 |
| Coconut milk powder | 1.000 | 0.74 |
| Garcinia cambogia | 0.600 | 0.44 |
| Phosphoric acid | 0.400 | 0.30 |
| Caralluma fimbriatta | 0.200 | 0.15 |
| N-oleayl-phosphatidyl- | 0.150 | 0.11 |
| ethanolamine | | |
| Rebiana A, | 0.150 | 0.11 |
| Coleus forskholii 10% extract | 0.05 | 0.04 |
| Sodium alginate | 0.050 | 0.04 |
| Ginger powder | 0.050 | 0.04 |
| Green tea extract, 50% ECGC | 0.050 | 0.04 |
| Vitamin B3 (as niacin) | 0.050 | 0.04 |
| TOTAL | 100.00 | 74.00 |

### EXAMPLE 4

| | % with respect to | Milliliters per |
|---|---|---|
| | the total mass of | 74 milliliter |
| INGREDIENTS | the composition | composition |
| Whey protein hydrolyzate | 12.000 | 8.88 |
| Cranberry juice from concentrate | 33.300 | 24.64 |
| Young Coconut water from | 30.000 | 22.20 |
| concentrate | | |
| Coconut milk power | 1.000 | 0.74 |
| Garcinia cambogia | 0.600 | 0.44 |
| Ascorbic acid | 0.400 | 0.30 |
| Nopal pad extract from powder | 21.95 | 16.24 |
| Caralluma fimbriatta, Gencor | 0.200 | 0.15 |
| Slimaluma | | |
| N-oleayl-phosphatidyl-ethanolamine | 0.100 | 0.07 |
| Rebiana A, | 0.150 | 0.11 |
| Coleus forskholii 10% extract | 0.05 | 0.04 |
| Guar gum | 0.050 | 0.04 |
| Ginger powder | 0.050 | 0.04 |
| Green tea extract, 50% ECGC | 0.100 | 0.07 |
| Vitamin B3 (as niacin) | 0.050 | 0.04 |
| TOTAL | 100.00 | 74.00 |

### EXAMPLE 5

| | % with respect to | Milliliters per |
|---|---|---|
| | the total mass of | 74 milliliter |
| INGREDIENTS | the composition | composition |
| Whey protein hydrolyzate | 12.000 | 8.88 |
| Dark Sweet Cherry juice from | 21.900 | 16.21 |
| concentrate | | |
| Young Coconut water from | 30.000 | 22.20 |
| concentrate | | |
| Strawberry juice from concentrate | 11.000 | 8.14 |
| Coconut milk powder | 1.000 | 0.74 |
| Malic acid | 0.800 | 0.59 |
| Garcinia cambogia | 0.600 | 0.44 |
| Nopal pad extract from powder | 21.95 | 16.24 |
| Caralluma fimbriatta | 0.200 | 0.15 |
| N-oleayl-phosphatidyl- | 0.100 | 0.07 |
| ethanolamine | | |
| Rebiana A, | 0.150 | 0.11 |
| Gum arabic | 0.050 | 0.04 |
| Ginger powder | 0.050 | 0.04 |
| Coleus forskholii 10% extract | 0.05 | 0.04 |
| Green tea extract, 50% ECGC | 0.100 | 0.07 |
| Vitamin B3 (as niacin) | 0.050 | 0.04 |
| TOTAL | 100.00 | 74.00 |

### EXAMPLE 6

| | % with respect to | Milliliters per |
|---|---|---|
| | the total mass of | 74 milliliter |
| INGREDIENTS | the composition | composition |
| Coconut water from concentrate | 30.000 | 22.20 |
| Nopal pad extract from powder | 21.95 | 16.24 |
| Whey protein hydrolyzate | 12.000 | 8.88 |
| Coconut milk powder | 1.000 | 0.74 |
| Garcinia cambogia extract | 0.600 | 0.44 |
| Caralluma fimbriatta extract | 0.200 | 0.15 |
| N-oleayl-phosphatidyl- | 0.100 | 0.07 |
| ethanolamine | | |
| Coleus forskhohlii extract | 0.050 | 0.04 |
| Ginger powder | 0.050 | 0.04 |
| Green tea extract | 0.100 | 0.07 |
| Coleus forskholii 10% extract | 0.05 | 0.04 |
| Vitamin B3 (as niacin) | 0.050 | 0.04 |
| Dark Sweet Cherry juice from | 19.100 | 14.13 |
| concentrate | | |
| Strawberry juice from | 12.800 | 9.47 |
| concentrate | | |
| Cocoa powder, Dutch process | 1.000 | 0.74 |
| Citric acid | 0.750 | 0.56 |
| Stevia extract | 0.150 | 0.11 |
| Xanthan gum | 0.050 | 0.04 |
| TOTAL | 100.00 | 74.00 |

Throughout this specification the word “comprise”, or variations such as “comprises” or “comprising”, will be understood to imply the inclusion of a component or group of components but not the exclusion of others.

The use of the expression “at least” or “at least one” suggests the use of one or more elements or ingredients or quantities, as the use may be in the embodiment of the invention to achieve one or more of the desired objects or results. Whenever a range of values is specified with the term “about”, it should be appreciated that mathematical precision is not contemplated.

While considerable emphasis has been placed herein on the particular features of this invention, it will be appreciated that various modifications can be made, and that many changes can be made in the preferred embodiments without departing from the principle of the invention. These and other modifications in the nature of the invention or the preferred embodiments will be apparent to those skilled in the art from the disclosure herein, whereby it is to be distinctly understood that the foregoing descriptive matter is to be interpreted merely as illustrative of the invention and not as a limitation.

What is claimed is:

1. A composition for oral administration for weight management; said composition comprising:
at least about ⅔ by weight of active ingredients; and
said composition being free of caffeine.

2. The composition according to claim 1, wherein said composition provides about 40 calories per 74 milliliters.

3. The composition according to claim 1, wherein about ⅓ by weight includes one or more of sweetening, flavoring and palatability ingredients.

4. The composition according to claim 1, wherein the composition is free of added sugar.

5. The composition according to claim 1, wherein the active ingredients include one or more of.
a) protein;
b) coconut water;
c) nopal extract;
d) Caralluma fimbriatta;
e) tea extract;
f) Garcinia cambogia extract;
g) N-oleayl-phosphatidyl-ethanolamine (OEA);
h) Coleus forshkohlii; and
i) vitamin B-3.

6. The composition according to claim 4, wherein the coconut water is from coconut water concentrate, the nopal is nopal pad juice from powder and the green tea extract includes approximately 50% epigallocatechin gallate (ECGC).

7. The composition according to claim 4, wherein the protein is whey protein hydrolyzate.

8. The composition according to claim 4, wherein the tea extract is green tea extract.

9. The composition according to claim 2, wherein the flavoring and palatability ingredients include one or more of:
a. juices from fruit;
b. juices from vegetables;
c. organic acids; and
d. emulsifying and suspending agents.

9. The composition according to claim 4, wherein nopal extract is present as an active ingredient, said nopal extract being in the form of one or more of:
Nopal freeze dried powder; and
Nopal juice.

10. The composition according to claim 1, wherein the composition provides about 8 grams of protein.

11. The composition according to claim 1, wherein the composition provides about 3 total grams of carbohydrates.

12. The composition according to claim 1, wherein one or more of the following ingredients are present in the following ranges each with respect to the total mass of the composition:
a. protein hydrolyzate in the range between 10% and 20%;
b. coconut water concentrate in the range between 1% and 35%;
c. nopal extract;
i. in a form of a nopal freeze dried powder in the range between 0.1% and 1%; or
ii. in a form of a nopal juice in the range between 15% and 30%;
d. Caralluma fimbriatta in the range between 0.1% and 1%;
e. tea extract in the range between 0.01% and 0.5%;
f. Coleus forskholii 10% extract in the range between 0.05 and 0.50%;
g. Garcinia cambogia extract in the range between 0.3% and 1%;
h. a mixture of N-oleayl-phosphatidyl-ethanolamine (OEA) and epigallocatechin gallate (EGCG) is in the range between 0.5% and 2%;
i. vitamin B-3 in the range between 0.01% and 0.5%;
j. coconut milk powder in the range of 0.5% and 2%;
k. citric acid in the range of 0.5% and 2%;
l. ginger powder in the range of 0.01% and 0.5%;
m. cherry juice concentrate in the range of 15% and 25%;
n. strawberry juice concentrate in the range of 10% and 15%;
o. chocolate powder in the range of 0.5% and 1.5%;
p. rebaudinoside in the range of 0.05% and 2%;
q. xanthan gum in the range of 0.02% and 1%.

13. The composition according to claim 1, including a sweetening ingredient.

14. The composition according to claim 13, wherein the sweetening ingredient is one or more of stevia, aspertame and sucralose.

15. The composition according to claim 1, wherein at least one active ingredient is selected from the group consisting of egg protein, whey protein and soy protein.

16. The composition of claim 1, said composition being formulated for oral administration into a dosage form selected from the group consisting of a powder, a tablet, a pill, a capsule, a liquid, a beverage, a suspension and an emulsion.

17. A method for weight management, said method comprising administering the composition as claimed in claim 1 to a subject to satiate craving for nutrition.

18. The method as claimed hi claim 17 in which the composition is administered in the amount of 74 ml.

19. The method as claimed in claim 17 in which the composition is administered between about ½ hour and 2 hours prior to the time of meal consumption.
